(19) United States
(12) Patent Application Publication
(10) Pub. No.: US 2009/0247535 A1
(43) Pub. Date: Oct. 1, 2009

(54) USE OF RANOLAZINE FOR NON-CARDIOVASCULAR DISORDERS

(75) Inventor: Geoffrey S. Pitt, Durham, NC (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: Duke University

(21) Appl. No.: 12/411,885

(22) Filed: Mar. 26, 2009

Related U.S. Application Data
(60) Provisional application No. 61/039,559, filed on Mar. 26, 2008.

(51) Int. Cl. A61K 31/495; A61P 25/08

(52) U.S. Cl. 514/252.12

(57) ABSTRACT

Disclosed is a method of treating a non-cardiovascular disorder characterized by an abnormal persistent sodium current in a subject in need of treatment thereof, said method comprising administering to the subject an effective amount of a compound of Formula (I):

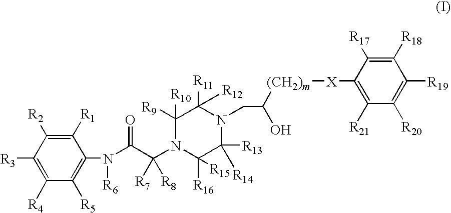

wherein:

m is 1 or 2;

R₁, R₂, R₃, R₄, and R₅ are each independently selected from the group consisting of hydrogen, halo, CF₃, CN, OR₂₂, SR₂₂, N(R₂₂)₂, S(=O)R₂₂, SO₂N(R₂₂)₂, C(═O)R₂₂, alkylamido, alkyl, alkenyl, morpholino, and pyrrolyl, wherein the alkyl substituent is optionally substituted with one substituent selected from the group consisting of OR₂₂, or R₂ and R₃ together or R₄ and R₅ together can be alkylene;

R₆, R₇, and R₈ are each independently selected from the group consisting of H and alkyl;

R₉, R₁₀, R₁₁, R₁₂, R₁₃, R₁₄, R₁₅, and R₁₆ are each independently selected from the group consisting of H and alkyl, or wherein one or more of R₉ and R₁₀, R₁₁ and R₁₂, R₁₃ and R₁₄, and R₁₅ and R₁₆ are together ═O;

R₁₇, R₁₈, R₁₉, R₂₀ and R₂₁ are each independently selected from the group consisting of H, halo, OR₂₂, alkyl, and aryl;

X is O or S; and

R₂₂ is selected from the group consisting of H and alkyl; or a pharmaceutically acceptable salt or a pharmaceutically acceptable ester thereof. The disorder can be epilepsy.

### RELATED APPLICATIONS

This application claims priority to U.S. Provisional Patent Application Ser. No. 61/093,559, filed Mar. 26, 2008, which is herein incorporated in its entirety.

### TECHNICAL FIELD

The presently disclosed subject matter relates to methods and compositions for treating non-cardiovascular disorders.

### BACKGROUND

Ranolazine and other substituted piperazine compounds, their pharmaceutically acceptable salts, and esters are disclosed in U.S. Pat. No. 4,567,264, herein incorporated by reference in its entirety, for use in the treatment of cardiovascular diseases, such as arrhythmias, variant and exercise-induced angina and myocardial infarction. See also, U.S. Pat. Nos. 5,506,229, and 6,677,336, each of which is also incorporated by reference herein in its entirety. Ranolazine is sold under the tradename RANEXA™ (CV Therapeutics, Palo Alto, Calif., United States of America) and has been approved for the treatment of angina (cardiac ischemia). However, it is believed that there have been no non-cardiovascular treatments disclosed in the art with respect to ranolazine and other substituted piperazine compounds,

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

Disclosed is a method of treating a non-cardiovascular disorder characterized by an abnormal persistent sodium current in a subject in need of treatment thereof, said method comprising administering to the subject an effective amount of a compound of Formula (I):

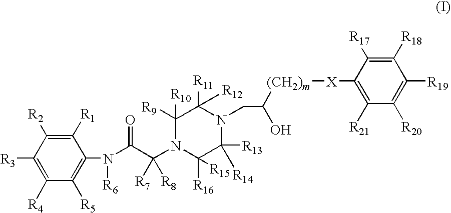

wherein:

m is 1 or 2;

R₁, R₂, R₃, R₄, and R₅ are each independently selected from the group consisting of hydrogen, halo, CF₃, CN, OR₂₂, SR₂₂, N(R₂₂)₂, S(═O)R₂₂, SO₂N(R₂₂)₂, C(═O)R₂₂, alkylamido, alkyl, alkenyl, morpholino, and pyrrolyl, wherein the alkyl substituent is optionally substituted with one substituent selected from the group consisting of OR₂₂, or R₂ and R₃ together or R₄ and R₅ together can be alkylene;

R₆, R₇, and R₈ are each independently selected from the group consisting of H and alkyl;

R₉, R₁₀, R₁₁, R₁₂, R₁₃, R₁₄, R₁₅, and R₁₆ are each independently selected from the group consisting of H and alkyl, or wherein one or more of R₉ and R₁₀, R₁₁ and R₁₂, R₁₃ and R₁₄, and R₁₅ and R₁₆ are together ═O;

R₁₇, R₁₈, R₁₉, R₂₀ and R₂₁ are each independently selected from the group consisting of H, halo, OR₂₂, alkyl, and aryl;

X is O or S; and

R₂₂ is selected from the group consisting of H and alkyl; or

a pharmaceutically acceptable salt or a pharmaceutically acceptable ester thereof.

In some embodiments the disorder is epilepsy. In some embodiments, X is O. In some embodiments, m is 1. In some embodiments, R₉, R₁₀, R₁₁, R₁₂, R₁₃, R₁₄, R₁₅, and R₁₆ are each H. In some embodiments, the compound is:

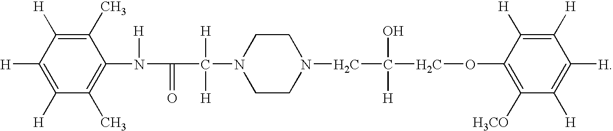

Also disclosed herein is a method of treating epilepsy in a subject in need of treatment thereof, said method comprising administering to the subject an effective amount of a compound of Formula (I):

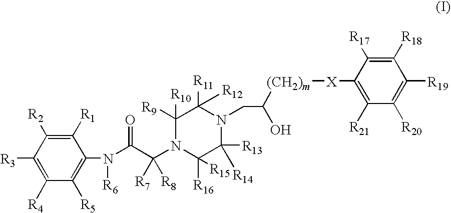

wherein:

m is 1 or 2;

R₁, R₂, R₃, R₄, and R₅ are each independently selected from the group consisting of hydrogen, halo, CF₃, CN, OR₂₂, SR₂₂, N(R₂₂)₂, S(═O)R₂₂, SO₂N(R₂₂)₂, C(═O)R₂₂, alkylamido, alkyl, alkenyl, morpholino, and pyrrolyl, wherein the alkyl substituent is optionally substituted with one substituent selected from the group consisting of OR₂₂, or R₂ and R₃ together or R₄ and R₅ together can be alkylene;

R₆, R₇, and R₈ are each independently selected from the group consisting of H and alkyl;

R₉, R₁₀, R₁₁, R₁₂, R₁₃, R₁₄, R₁₅, and R₁₆ are each independently selected from the group consisting of H and alkyl, or wherein one or more of R₉ and R₁₀, R₁₁ and R₁₂, R₁₃ and R₁₄, and R₁₅ and R₁₆ are together ═O;

R₁₇, R₁₈, R₁₉, R₂₀ and R₂₁ are each independently selected from the group consisting of H, halo, OR₂₂, alkyl, and aryl;

X is O or S; and

R₂₂ is selected from the group consisting of H and alkyl; or

a pharmaceutically acceptable salt or a pharmaceutically acceptable ester thereof.

In some embodiments, the compound is:

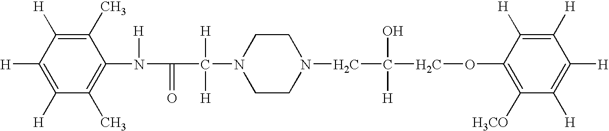

It is an object of the presently disclosed subject matter to provide methods and compositions for treating non-cardiovascular disorders.

An object of the presently disclosed subject matter having been stated above, other objects and advantages will become apparent to those of ordinary skill in the art after a study of the following description of the presently disclosed subject matter.

### DETAILED DESCRIPTION

Ranolazine and other substituted piperazine compounds, their pharmaceutically acceptable salts, and esters are disclosed in U.S. Pat. No. 4,567,264, herein incorporated by reference in its entirety, for use in the treatment of cardiovascular diseases, such as arrhythmias, variant and exercise-induced angina and myocardial infarction. See also, U.S. Pat. Nos. 5,506,229, and 6,677,336, each of which is also incorporated by reference herein in its entirety. Ranolazine is sold under the tradename RANEXA™ (CV Therapeutics, Palo Alto, Calif., United States of America) and has been approved for the treatment of angina (cardiac ischemia).

The previously postulated mechanism of action for ranolazine involves a perturbation of myocyte metabolism, specifically partial inhibition of fatty acid oxidation. However, this concept has been largely discounted. More recently, ranolazine has been recognized to block the “late” or “persistent” sodium current. By this mechanism, ranolazine is thought to decrease calcium influx through the sodium-calcium exchanger and thereby alter cardiac muscle dynamics. See Scheme 1. Ranolazine has been proven safe (and somewhat effective for cardiac ischemia) in several large clinical trials.

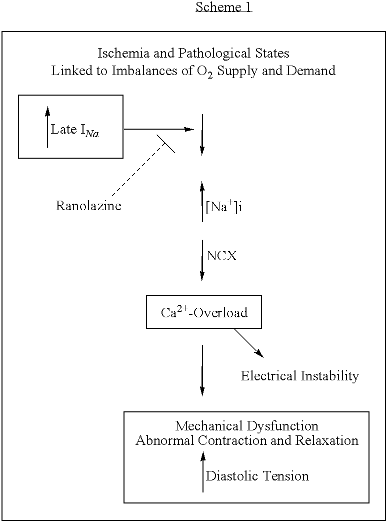

An increase in the persistent sodium current within the heart is arrhythmogenic. In fact, inherited mutations that cause increased persistent sodium current underlie one form of cardiac arrhythmia called Long QT Syndrome type 3. Thus, the blockade of this persistent sodium current could be protective against arrhythmias and protection against arrhythmias has been found in the clinical trials looking at angina, suggesting that the effects on the persistent sodium current might be clinically significant.

The atomic structure of the voltage-gated sodium channel's C-terminus has been solved. The sodium channel C-terminus is a region involved in channel inactivation and is a region in which some of the epileptogenic mutations reside. The cardiac and primary neuronal isoforms (those that have been connected with inherited forms of epilepsy) are highly homologous in this region. Thus, an aspect of the presently disclosed subject matter is that the central tenets of sodium channel inactivation are similar for cardiac and neuronal sodium channels. Accordingly, another aspect of the presently disclosed subject matter is that ranolazine (i.e., (±)-N-(2,6-dimethylphenyl)-4-[2-hydroxy-3-(2-methoxyphenoxy)-propyl]-1-piperazine-acetamide) has similar effects on neuronal sodium channels as it does on cardiac sodium channels, namely the blockade of the persistent sodium current.

The presently disclosed subject matter provides a method of treating a non-cardiovascular disorder characterized by an abnormal persistent sodium current in a subject in need of treatment thereof, the method comprising administering an effective amount of a substituted piperazine compound. In some embodiments, the substituted piperazine is a compound of Formula (i):

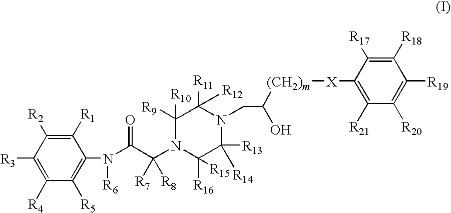

wherein:

m is 1 or 2;

R₁, R₂, R₃, R₄, and R₅ are each independently selected from the group consisting of hydrogen, halo, CF₃, CN, OR₂₂, SR₂₂, N(R₂₂)₂, S(═O)R₂₂, SO₂N(R₂₂)₂, C(═O)R₂₂, alkylamido, alkyl, alkenyl, morpholino, and pyrrolyl, wherein the alkyl substituent is optionally substituted with one substituent selected from the group consisting of OR₂₂, or R₂ and R₃ together or R₄ and R₅ together can be alkylene;

R₆, R₇, and R₈ are each independently selected from the group consisting of H and alkyl;

R₉, R₁₀, R₁₁, R₁₂, R₁₃, R₁₄, R₁₅, and R₁₆ are each independently selected from the group consisting of H and alkyl, or one or more of R₉ and R₁₀, R₁₁ and R₁₂, R₁₃ and R₁₄, and R₁₅ and R₁₆ are together ═O;

R₁₇, R₁₈, R₁₉, R₂₀ and R₂₁ are each independently selected from the group consisting of H, halo, OR₂₂, alkyl, and aryl;

X is O or S; and

R₂₂ is selected from the group consisting of H and alkyl; or

a pharmaceutically acceptable salt or a pharmaceutically acceptable ester thereof.

Non-cardiovascular disorders characterized by abnormal levels of persistent sodium current include, but are not limited to neuropathies; behavioral disorders and dementia; and movement and neurodegenerative diseases. In some embodiments, the disorder is epilepsy. Abnormal sodium current activity also can also be related to neurodegenerative disorders such as, without limitation, Parkinson's disease, Alzheimer's disease, Huntington's disease, amyotrophic lateral sclerosis and multiple sclerosis.

In some embodiments, the presently disclosed subject matter provides a method of treating epilepsy in a subject in need of treatment thereof, the method comprising administering to the subject an effective amount of a substituted piperazine compound. In some embodiments, the substituted piperazine is a compound of Formula (I). The epilepsy can be caused by a genetic defect (i.e., be a type of inherited epilepsy) or can be the result of metabolic abnormalities, such as low blood glucose or alcohol. The epilepsy can be caused by head injury or brain tumor. The epilepsy can be idiopathic or can be a seizure order of an unknown cause.

In some embodiments, X is O. In some embodiments, m is 3. In some embodiments, R₉-R₁₆ are each H. In some embodiments, the compound of Formula (I) is ranolazine. In some embodiments, the subject is a human subject. In particular, it is believed that ranolazine and other substituted piperazines of Formula (I) can be used to provide an effective treatment for seizures that differs in specific mechanism of action from currently available anti-epilepsy agents.

Ranolazine and other substituted piperazine compounds of Formula (I) can be screened for activity against the late sodium current from channels derived from the SCN1A and SCN2A genes in standard electrophysiology assays known in the art. Ranolazine and other substituted piperazine compounds of Formula (I) can be screened for anti-convulsant activity in a number of animal models known in the art. For example, drosophila and vertebrate models are available. The testing of ranolazine and other substituted piperazine compounds can include testing the compound's ability to penetrate the blood-brain barrier.

As used herein the term “alkyl” can refers to C₁₋₂₀ inclusive, linear (i.e., “straight-chain”), branched, or cyclic hydrocarbon chains, including for example, methyl, ethyl, propyl, isopropyl, butyl, isobutyl, tert-butyl, pentyl, hexyl, heptyl, and octyl. In some embodiments, alkyl refers to a C₁₋₁₅alkyl group. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “lower alkyl” refers, in particular, to C₁₋₈, C₁₋₅, or C₁₋₄ straight-chain or branched chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “alkenyl” refers to C₁-C₂₀ partially or, in some embodiments, completely unsaturated hydrocarbon or carbon chains. Thus, “alkenyl” can refer to ethenyl, propenyl, butenyl, pentenyl, hexenyl, octenyl, butadienyl, propynyl, butynyl, pentynyl, hexynyl, heptynyl, and allenyl groups. Alkenyl groups can optionally be substituted by one or more alkyl group substituent.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. In some embodiments, alkylene refers to a —C═C—C═C— or a —O—CH₂—O— group.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

“Alkoxyl” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangably with “alkoxyl”.

The term alkylamido refers to a —N(R)—C(═O)—R′ group wherein R is H or alkyl and R′ is alkyl, as defined herein.

The term “carbonyl” refers to the —(C═O)— group.

The terms “halo”, “halide”, or “halogen” as used herein refer to fluoro, chloro, bromo, and iodo groups.

The term “hydroxyl” refers to the —OH group.

The term “thio” refers to a compound wherein a carbon or oxygen atom is replaced by a sulfur atom. Thus, alkylthio refers to a RS— group, wherein R is alkyl as previously defined herein.

The term “alkyl sulfinyl” refers to a —S(═O)—R group, wherein R is alkyl.

The term “alkyl sulfonyl” refers to a —S(═O)₂—R group wherein R is alkyl.

When the term “independently selected” is used, the substituents being referred to (e.g., R groups, such as groups R₁ and R₂, or groups Z and X), can be identical or different. For example, both R₁ and R₂ can be substituted alkyls, or R₁ can be hydrogen and R₂ can be a substituted alkyl, and the like.

A named “R”, “X”, or “Z” group will generally have the structure that is recognized in the art as corresponding to a group having that name, unless specified otherwise herein. For the purposes of illustration, certain representative “R,” “X,” and “Z” groups as set forth above are defined below. These definitions are intended to supplement and illustrate, not preclude, the definitions that would be apparent to one of ordinary skill in the art upon review of the present disclosure.

The term “pharmaceutically acceptable salt” can refer to any salt that retains the biological effectiveness and properties of the free base compound and which is not biologically or otherwise undesirable. In some embodiments, the salt is pharmaceutically acceptable in humans. In some embodiments, the pharmaceutically acceptable salt is a pharmaceutically acceptable acid addition salt, formed with an inorganic acid, such as hydrochloric acid, hydrobromic acid, sulfuric acid, nitric acid, phosphoric acid and the like, or formed with an organic acid, such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicyclic acid, and the like.

“Pharmaceutically acceptable ester” includes those containing the alkanoyloxy group —O—C(═O)-Z, wherein Z is an alkyl group containing 1 to 12 carbon atoms, which is attached to carbon atom 2 of the propylene or butylene linkage of the compound of Formula (I) instead of hydroxyl (i.e., the hydroxyl group of Formula (I) has been esterified). The group Z can be, for example, methyl, ethyl, butyl, hexyl, octyl, dodecyl, and the like. The presently disclosed subject matter provides compounds of Formula (I) which are both esters and at the same time are pharmaceutically acceptable salts.

According to the presently disclosed subject matter the compound of Formula (I) can be provided as a mixture of stereoisomers (e.g., as a racemic mixture) or as a pure stereoisomer.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of treating a non-cardiovascular disorder characterized by an abnormal persistent sodium current in a subject in need of treatment thereof, said method comprising administering to the subject an effective amount of a compound of Formula (I): 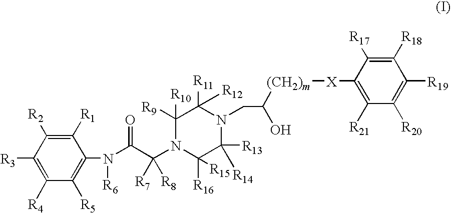
wherein:
m is 1 or 2;
R₁, R₂, R₃, R₄, and R₅ are each independently selected from the group consisting of hydrogen, halo, CF₃, CN, OR₂₂, SR₂₂, N(R₂₂)₂, S(═O)R₂₂, SO₂N(R₂₂)₂, C(═O)R₂₂, alkylamido, alkyl, alkenyl, morpholino, and pyrrolyl, wherein the alkyl substituent is optionally substituted with one substituent selected from the group consisting of OR₂₂, or R₂ and R₃ together or R₄ and R₅ together can be alkylene;
R₆, R₇, and R₈ are each independently selected from the group consisting of H and alkyl;
R₉, R₁₀, R₁₁, R₁₂, R₁₃, R₁₄, R₁₅, and R₁₆ are each independently selected from the group consisting of H and alkyl, or wherein one or more of R₉ and R₁₀, R₁₁ and R₁₂, R₁₃ and R₁₄, and R₁₅ and R₁₆ are together ═O;
R₁₇, R₁₈, R₁₉, R₂₀ and R₂₁ are each independently selected from the group consisting of H, halo, OR₂₂, alkyl, and aryl;
X is O or S; and
R₂₂ is selected from the group consisting of H and alkyl; or
a pharmaceutically acceptable salt or a pharmaceutically acceptable ester thereof.

2. The method of claim 1, wherein the disorder is epilepsy.

3. The method of claim 1, wherein X is O.

4. The method of claim 1, wherein m is 1.

5. The method of claim 1, wherein R₉, R₁₀, R₁₁, R₁₂, R₁₃, R₁₄, R₁₅, and R₁₆ are each H.

6. The method of claim 1, wherein the compound is:

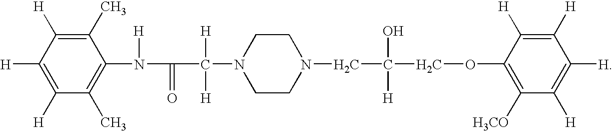

7. A method of treating epilepsy in a subject in need of treatment thereof, said method comprising administering to the subject an effective amount of a compound of Formula (I): 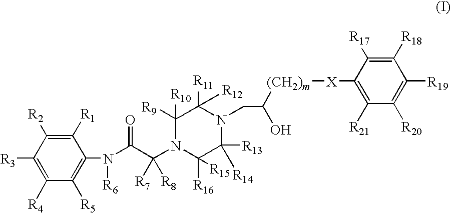
wherein:
m is 1 or 2;
R₁, R₂, R₃, R₄, and R₅ are each independently selected from the group consisting of hydrogen, halo, CF₃, CN, OR₂₂, SR₂₂, N(R₂₂)₂, S(═O)R₂₂, SO₂N(R₂₂)₂, C(═O)R₂₂, alkylamido, alkyl, alkenyl, morpholino, and pyrrolyl, wherein the alkyl substituent is optionally substituted with one substituent selected from the group consisting of OR₂₂, or R₂ and R₃ together or R₄ and R₅ together can be alkylene;
R₆, R₇, and R₈ are each independently selected from the group consisting of H and alkyl;
R₉, R₁₀, R₁₁, R₁₂, R₁₃, R₁₄, R₁₅, and R₁₆ are each independently selected from the group consisting of H and alkyl, or wherein one or more of R₉ and R₁₀, R₁₁ and R₁₂, R₁₃ and R₁₄, and R₁₅ and R₁₆ are together ═O;
R₁₇, R₁₈, R₁₉, R₂₀ and R₂₁ are each independently selected from the group consisting of H, halo, OR₂₂, alkyl, and aryl;
X is O or S; and
R₂₂ is selected from the group consisting of H and alkyl; or
a pharmaceutically acceptable salt or a pharmaceutically acceptable ester thereof.

8. The method of claim 7, wherein the compound is: [chemical structure]
